(19) United States
(12) Patent Application Publication
(10) Pub. No.: US 2026/0157963 A1
(43) Pub. Date: Jun. 11, 2026

(54) MIXTURE, AQUEOUS SOLUTION CONTAINING THE MIXTURE, AND USES OF THE AQUEOUS SOLUTION

(71) Applicant: FRAUNHOFER-GESELLSCHAFT ZUR FÖRDERUNG DER ANGEWANDTEN FORSCHUNG E.V., München (DE)

(72) Inventors: Raphael THIERMANN, Mainz (DE); Regina BLEUL, Mainz (DE); Ruben ROSENCRANTZ, Golm (DE); Sany CHEA, Golm (DE)

(73) Assignee: FRAUNHOFER-GESELLSCHAFT ZUR FÖRDERUNG DER ANGEWANDTEN FORSCHUNG E.V., München (DE)

(21) Appl. No.: 18/842,216

(22) PCT Filed: Feb. 24, 2023
(86) PCT No.: PCT/EP2023/054673

§ 371 Date: Aug. 28, 2024

(30) Foreign Application Priority Data
Mar. 1, 2022 (DE) 10 2022 202 099.5

(51) Int. Cl. A61K 9/107; A61K 9/00; A61K 47/26; A61K 47/32; A61K 47/42; C08L 33/26

(52) U.S. Cl. CPC A61K 9/107; A61K 9/0004; A61K 47/26; A61K 47/32; A61K 47/42; C08L 33/26

(57) ABSTRACT

The invention relates to a mixture containing a first polymer and a second polymer. The first polymer and the second polymer each comprise at least six nucleobases, wherein each nucleobase is covalently bonded to one monomer of the relevant polymer. The at least six nucleobases of the second polymer are complementary to the at least six nucleobases of the first polymer. Because of their mutually complementary nucleobases, the two polymers are suitable for forming micelles or vesicles (preferably vesicles) at temperatures of ≤37° C. and are not suitable for forming micelles or vesicles at temperatures of >37° C. According to the invention, an aqueous solution containing the mixture according to the invention is also provided and uses of the aqueous solution are proposed.

A mixture is provided which comprises a first polymer and a second polymer. The first polymer and the second polymer each comprise at least six nucleobases, wherein one nucleobase is covalently bonded to one monomer of the respective polymer, respectively. The at least six nucleobases of the second polymer are complementary to the at least six nucleobases of the first polymer. The two polymers are capable, via their mutually complementary nucleobases, at a temperature of ≤37° C., to assemble to micelles or vesicles (preferably vesicles), and, at a temperature of >37° C., to not assemble to micelles or vesicles. According to the invention, an aqueous solution comprising the mixture according to the invention is further provided and uses of the aqueous solution are proposed.

Temperature-controlled release of substances may play an important role in many fields of application. These range from medical applications (e.g., tumor treatment) and cosmetic applications to technical processes for the release of coolants or lubricants.

Stimuli-responsive active-substance delivery systems are intended to help release their active substance in a spatially, temporally and dose-controlled manner. The control of the release can be endogenous, i.e., triggered by conditions at the target site (e.g., pH, enzyme concentrations, redox gradients), or exogenous, i.e., triggered by an external stimulus (e.g., temperature change, magnetic field, ultrasound, light treatment).

Thermoresponsive systems are generally based on a temperature-dependent, preferably sharp, non-linear change in the physicochemical properties of at least one component of the carrier material, which leads to the release of the enclosed active substance.

Polymer-based, thermoresponsive active-substance transporters based on so-called UCST polymers (upper critical solution temperature polymers) are known in the prior art. The UCST polymer poly-2-oxazoline can in principle be used for active-substance transport, but is not suitable for targeted active-substance release in living organisms with a body temperature of 37° C., as its switching temperature (release temperature) is already 30° C. (Hoogenboom, R. et al., Soft Matter, 2009, vol. 5. p. 3590-3592). The UCST polymer poly(AAm-co-AN)-g-PEG) is known to form vesicles in aqueous solution that can be loaded with the active substance doxorubicin. However, the vesicles exhibit a temperature-dependent release of doxorubicin in a very wide temperature range between 4° C. and 43° C., which also makes them unsuitable for targeted active-substance release in organisms with a body temperature of 37° C. (Li, W. S. et al., Angew. Chem. Int. ed., 2015, vol. 54, p. 3126-3131).

With the known UCST polymer systems, it has not yet been possible to precisely set a sharp switching limit in order to achieve safe and sensitive release behavior under in vivo conditions.

Ideally, the switching temperature of thermoresponsive systems as active-substance carriers should be very close to the physiological body temperature of 37° C., so that no unspecific release occurs in healthy tissue, i.e., already at 37° C. On the other hand, however, even a small local temperature increase of a few Kelvin (e.g., to approx. 40-42° C.) should ideally be sufficient to induce the release of the active substance, since if the necessary switching temperatures are too high, a very high heating of body tissue is required, which can also damage certain healthy tissue in the body (e.g., neighboring tissue of a tumor) in a desired manner.

In the prior art, a DNA-based active-substance delivery system is already known in which mesoporous silica particles are covalently bound to single-stranded DNA and this single-stranded DNA hybridizes to a complementary single-stranded DNA covalently bound to iron oxide nanoparticles, thereby sealing the pores of the mesoporous silica particles (Ruiz-Hernandez, E. et al., ACS Nano, 2011, vol. 5, p. 1259-1266). Melting the hybridization of the DNA by raising the temperature then allows the pores of the mesoporous silica particles to open and release an active substance trapped inside. However, this DNA-based active-substance release system is complex to manufacture, allows only a relatively low active-substance loading in relation to the total weight of the system and has a relatively wide active-substance release temperature range.

Based on this, it was the object of the present invention to provide an active-substance release system (or a mixture) which does not have the disadvantages of the prior art. In particular, the active-substance system should be easy and economical to produce on an industrial scale, allow a high active-substance loading relative to the total weight of the system and enable active substances to be released in the narrowest possible temperature range at a temperature of >37° C.

The object is achieved by the method having the features of claim 1, the electrode having the features of claim 12, and the use having the features of claim 15. The dependent claims describe advantageous developments.

In accordance with the invention, a mixture is provided, comprising
a) a first polymer comprising at least six nucleobases, wherein one nucleobase is covalently bonded to one monomer of the polymer, respectively; and
b) a second polymer comprising at least six nucleobases, wherein one nucleobase is covalently bonded to one monomer of the polymer, respectively, wherein the at least six nucleobases of the second polymer are complementary to the at least six nucleobases of the first polymer;
   characterized in that the first polymer and the second polymer are capable, via their mutually complementary nucleobases, in an aqueous solution
   at a temperature of ≤37° C., by forming hydrogen bonds between their complementary nucleobases, to assemble to micelles or vesicles (i.e., to form micelles or vesicles or to be present as micelles or vesicles), and
   at a temperature of >37° C., by the absence of hydrogen bonds between their complementary nucleobases, to not assemble to micelles or vesicles (i.e., not to form micelles or vesicles or not to be present as micelles or vesicles).

The self-assembly of the polymer components of the mixture at a temperature of ≤37° C. is based on the formation of hydrogen bonds between the individual complementary nucleobases of the two polymers, which causes micelle formation or vesicle formation of the two polymers. If the melting temperature of the bond between the complementary nucleobases is exceeded at a temperature of >37° C., the bond between the two polymers is lost, i.e., the two polymers dissociate and enter into a non-micellar or non-vesicular state.

The micelles or vesicles dissolve in the process. If an active substance was trapped in the micelles or vesicles, it is released. If the temperature range of the melting of the hydrogen bonds is narrow, i.e., only a few Kelvin wide, a temperature difference of a few Kelvin above 37° C. (e.g., a temperature jump from 37° C. to 40° C.) can already cause a release of active substance.

The mixture according to the invention has the advantage that a release of active substances is possible in a temperature range of >37°, in particular in a narrow temperature range above 37° C. (e.g., 1 K to 5 K, preferably 1 K to 3 K, above 37° C.). Furthermore, the mixture can be produced easily and economically on an industrial scale. In addition, the mixture allows a relatively high active-substance loading if the first and second polymers form vesicles as active-substance carriers. Vesicles provide a large interior space for loading with active substance and the polymers that form the vesicles only have a small volume and therefore a low weight in relation to this large interior space. Consequently, it is preferred that the first and second polymers form vesicles. If the first and second polymers form micelles, a certain, but smaller, active-substance loading is also possible (especially with more hydrophobic active substances).

This property makes the mixture according to the invention particularly useful for thermally induced switching in the body of a living organism with a body temperature of 37° C., since it is particularly advantageous here if the temperature required for switching is not too high above 37° C. (reason: less undesirable thermally induced damage to the tissue occurs). For example, it is also known that a cancerous tumor in the body can have a temperature that is 1 K to 2 K higher than that of the surrounding body tissue (which is 37° C.), which means that the mixture according to the invention can be used to release an active substance in the body specifically at the site of the tumor.

The formation of vesicles is preferred because vesicles provide a larger interior space for loading with an active substance and thus allow loading with larger active-substance molecules or a larger quantity of smaller active-substance molecules. It is thus preferred that the first polymer and the second polymer, via their mutually complementary nucleobases, in an aqueous solution, are capable, at a temperature of ≤37° C., by forming hydrogen bonds between their complementary nucleobases, to assemble to vesicles (i.e., to form vesicles or be present as vesicles), and, at a temperature of >37° C., due to the absence of hydrogen bonds between their complementary nucleobases, to not assemble to vesicles (i.e., not to form vesicles or not be present as vesicles).

In a preferred embodiment, the mixture comprises a magnesium salt (e.g., MgCl₂) and/or a magnesium complex, particularly preferably in a concentration of 10 to 30 mM. The presence of magnesium may be advantageous for the formation of vesicles or micelles if the first polymer and/or second polymer comprise at least one monomer comprising a phosphate residue (e.g., a ribose-5-phosphate residue).

In a preferred embodiment, the mixture comprises an active substance, wherein the active substance is preferably selected from the group consisting of medicinally active substances, cosmetic substances, sensor substances, lubricants, coolants, adhesives and combinations thereof. The active substance is particularly preferably selected from the group consisting of medicinally active substances, fragrances, antiperspirants, UV protectants, coolants, lubricants, adhesives and combinations thereof. The advantage here is that the active substance can be enclosed in an interior of the micelles or vesicles formed from the two polymers of the mixture according to the invention at a temperature of ≤37° C. in aqueous solution.

In a further optional embodiment, the first polymer and/or the second polymer is a linear (i.e., unbranched) polymer. Alternatively, the first polymer and/or the second polymer is a non-linear (i.e., branched) polymer. Here, the first polymer and/or the second polymer can have at least one branch, optionally two branches. If the first polymer and/or second polymer has one or two branches, it may be a modified 3-arm PEG or modified 4-arm PEG (e.g., with an average molar mass of 1000 to 6000 g/mol, preferably 2000 g/mol to 5000 g/mol), which has monomers on at least two arms of the PEG, preferably on at least three arms of the PEG (optionally on four arms of the PEG in the case of a 4-arm PEG), wherein one monomer is covalently bonded to one nucleobase, respectively (preferably at one end of each of the arms). The decisive factor is that the 3-arm PEG or 4-arm PEG has at least six nucleobases, wherein one nucleobase is covalently bonded to one monomer of the polymer, respectively. In one arm of the 3-arm PEG or the 4-arm PEG, for example, at least four or six such monomers may be present and in a second arm of this PEG, for example, four or six such monomers may be present. It is possible that the other arms of the 3-arm PEG or 4-arm PEG have no such monomers, i.e., have no monomers bonded to a nucleobase (nucleobase-free monomers).

The first polymer may comprise or consist of at least 20, preferably at least 50, particularly preferably at least 100, very particularly preferably at least 200, in particular at least 500 monomers. Furthermore, the second polymer may comprise or consist of at least 20, preferably at least 50, particularly preferably at least 100, very particularly preferably at least 200, in particular at least 500 monomers. The first and/or second polymer may comprise a maximum of 600 monomers, optionally a maximum of 500 monomers.

The first polymer may comprise at least ten, preferably at least twelve, particularly preferably at least 14, in particular at least 16, optionally a maximum of 18, nucleobases, wherein one nucleobase is covalently bonded to one monomer of the first polymer, respectively. The second polymer may comprise at least ten, preferably at least twelve, particularly preferably at least 14, in particular at least 16, optionally a maximum of 18, nucleobases, wherein one nucleobase is covalently bonded to one monomer of the second polymer, respectively, wherein the nucleobases of the second polymer are complementary to the nucleobases of the first polymer. The more nucleobases which are complementary to each other comprised by the first and second polymer, the higher is the melting point of the hybridized nucleobases, i.e., the higher above >37° C. is the temperature at which the micelles and/or vesicles disassemble.

It is possible for the first polymer to have a maximum of 16, preferably a maximum of 14, particularly preferably a maximum of 12, optionally a maximum of 10, nucleobases. Furthermore, it is possible that the second polymer has a maximum of 16, preferably a maximum of 14, particularly preferably a maximum of 12, optionally a maximum of 10, nucleobases. The fewer nucleobases which are complementary to each other and are comprised by the first and second polymer, the lower the melting point of the hybridized nucleobases, i.e., the closer to >37° C. is the temperature at which the micelles and/or vesicles disassemble.

In the first polymer, a maximum of three monomers, preferably a maximum of two monomers, particularly preferably a maximum of one monomer, in particular no monomer, which is not in each case covalently bonded to a nucleobase, i.e., which is not in each case bonded to a nucleobase-free monomer, may be arranged between two successive monomers each bonded to one nucleobase, respectively. The term “nucleobase-free monomer” refers to a monomer which itself (i.e., considered as an isolated building block in the polymer) has no bond to a nucleobase. In other words, the nucleobase-free monomers only have a bond to a nucleobase (indirectly) via neighboring monomers of the polymer (which can then be regarded as “spacers”). Furthermore, in the second polymer, a maximum of three monomers, preferably a maximum of two monomers, particularly preferably a maximum of one monomer, in particular no monomer, which is not in each case covalently bonded to a nucleobase, i.e., which is not in each case bonded to a nucleobase-free monomer, may be arranged between two successive monomers each bonded to one nucleobase, respectively. If no nucleobase-free monomer is arranged between two consecutive, nucleobase-bonded monomers, the nucleobases are bound to sequentially successive monomers of the polymer. Such an arrangement of the nucleobases along the polymer chain has the advantage that a reversible binding mechanism is created over the shortest possible length, which can, depending on the ambient temperature, reversibly bind the first polymer and the second polymer to each other (T ≤37° C.) or release them from each other (T >37° C.).

The first polymer and/or second polymer may comprise at least one monomer or oligomer (preferably at least one monomer, particularly preferably exactly one monomer) which is not covalently bonded to a nucleobase in each case (i.e., which is a nucleobase-free monomer) and which is covalently bonded to at least one further polymer (optionally two further polymers, such as in a 3-arm PEG or 4-arm PEG). The further polymer is preferably suitable for effecting phase separation after assembly of the first and second polymers in an aqueous medium. The further polymer may comprise or consist of polyethylene glycol.

The nucleobases of the first polymer may be nucleobases comprising or consisting of cytosine, isocytosine and/or adenine, preferably cytosine. Furthermore, the nucleobases of the second polymer are nucleobases comprising or consisting of guanine, isoguanine and/or thymine, preferably guanine. The combination of cytosine or isocytosine (first polymer) and guanine or isoguanine (second polymer) is preferred, since the cytosine-guanine bond (or isocytosine-isoguanine bond), which is established by three hydrogen bonds, is stronger than the adenine-thymine bond, which is established by only two hydrogen bonds. The use of cytosine, guanine, isocytosine and isoguanine thus has the advantage that a smaller number of nucleobases is needed to set a certain (dis)assembly temperature than when adenine and thymine are used. Consequently, the reversible binding mechanism can be established over a shorter length along the first and second polymers.

The nucleobases of the first polymer may be bonded to the monomer of the first polymer via a ribose residue, wherein preferably the nucleobase is bonded to the C1 atom of the ribose residue and the monomer is bonded to the C5 atom of the ribose residue. Furthermore, the nucleobases of the second polymer may each be bonded to the one monomer of the first polymer via a ribose residue, wherein preferably the nucleobase is bonded to the C1 atom of the ribose residue and the monomer is bonded to the C5 atom of the ribose residue. The advantage of the bond via a ribose residue is that the ribose residue is relatively hydrophilic and can therefore improve the water solubility of the first and/or second polymer.

The monomers of the first polymer and/or second polymer, each bonded to one nucleobase, may each comprise or consist of a residue selected from the group consisting of amino acid residue, sugar residue, ribose phosphate residue, methacrylamide residue, and combinations thereof. The residue is preferably selected from the group consisting of ribose phosphate residue, methacrylamide residue and combinations thereof, particularly preferably selected from the group consisting of ribose-5-phosphate residue, N-(3-aminopropyl)-methacrylamide residue and combinations thereof. The advantage of the ribose-5-phosphate residue and the N-(3-aminopropyl)-methyacrylamide residue is that they are relatively hydrophilic and can therefore improve the water solubility of the first and/or second polymer.

Furthermore, the monomers of the first polymer and/or second polymer, which are each bonded to one nucleobase, can form a homopolymer. The homopolymer is preferably selected from the group consisting of polypeptide, polysaccharide, polyribose phosphate, polymethacrylamide and combinations thereof. Particularly preferred is the homopolymer selected from the group consisting of poly-ribose phosphate, polymethacrylamide and combinations thereof. In particular, the homopolymer is selected from the group consisting of poly-ribose-5-phosphate, poly-(N-(3-aminopropyl)-methacrylamide and combinations thereof. The advantage is that these homopolymers are relatively hydrophilic and can therefore improve the water solubility of the first and/or second polymer.

Furthermore, the monomers of the first polymer and/or second polymer, which are each bonded to one nucleobase, may be covalently bonded to the nucleobase via a bond (optionally via a ribose residue) selected from the group consisting of ester bond, thioester bond, ether bond and amide bond. Preferably, the bond is an amide bond, particularly preferably an amide bond to a ribose residue that is covalently bonded to the nucleobase. In particular, the bond is an amide bond to a C5 atom of a ribose residue that is covalently bonded to the nucleobase at the C1 atom of the ribose residue. The amide bond is advantageous because it is a relatively stable bond in an aqueous environment.

Furthermore, it is preferred that the first polymer and second polymer comprise the nucleobases, each of which is covalently bonded to one monomer of the polymer, at a first end of the polymer, at a first end of the polymer and at a second end of the polymer, or in the middle of the polymer. In other words, the nucleobases along the polymer chain occur only in that particular portion (or region or block) of the polymer and do not occur in a portion (or region or block) of the polymer other than that portion (or region or block) of the polymer.

For example, if the nucleobases are arranged at a first end of the polymer, each monomer at a second end of the polymer would have no attached nucleobase, i.e., only nucleobase-free monomers would be present at the second end. If, for example, the nucleobases are arranged at a first end of the polymer and at a second end of the polymer, each monomer in the middle of the polymer would not have a bound nucleobase, i.e., only nucleobase-free monomers would be present in the middle. For example, if the nucleobases are arranged in the middle of the polymer, each monomer at the beginning and at the end of the polymer (i.e., in both directions away from the middle of the polymer) would have no attached nucleobase, i.e., only nucleobase-free monomers would be present in these portions. The different portions (regions or blocks) of the polymer can therefore have different physical and/or chemical properties. For example, the portions of the first and/or second polymer of which the monomers are not each bonded to a nucleobase (portions with nucleobase-free monomers) exhibit a higher degree of freedom at a temperature of ≤37° C., since these regions are not involved in the hybridization of the two polymers, i.e., these portions do not undergo “immobilization” as a result of the hybridization. The longer this portion with nucleobase-free monomers of the first and/or second polymer is, the more strongly it determines the chemical and physical properties of the micelles or vesicles assembled from the two polymers.

Monomers of the first polymer and/or second polymer, each of which is not bonded to a nucleobase (nucleobase-free monomers), may each comprise or consist of a residue selected from the group consisting of (N-(2-hydroxypropyl)methacrylamide residue, vinylamine residue, butadiene residue, ethylene oxide residue, acrylic acid residue, amino acid residue (e.g., glutamic acid residue and/or lysine residue), sugar residue and combinations thereof. Preferably, these monomers comprise or consist of a (N-(2-hydroxypropyl)methacrylamide residue and/or an ethylene oxide residue, since these monomers have good water solubility and can thus provide good water solubility of the first and/or second polymer.

Monomers of the first polymer and/or second polymer, each of which is not bonded to a nucleobase (nucleobase-free monomers), may form a homopolymer, preferably a homopolymer selected from the group consisting of poly-(N-(2-hydroxypropyl)methacrylamide, polyvinylamine, polyethylene oxide, polypeptide (e.g., polyglutamic acid and/or polylysine), polysaccharide, and combinations thereof, particularly preferably poly-(N-(2-hydroxypropyl)methacrylamide. The formation of poly-(N-(2-hydroxypropyl)methacrylamide (pHPMA) and/or polyethylene oxide is advantageous because it has good water solubility and can thus provide good water solubility of the first and/or second polymer. Furthermore, monomers of the first polymer and/or second polymer, each of which is not bonded to a nucleobase (nucleobase-free monomers), may form a block copolymer, preferably a block copolymer selected from the group consisting of polybutadiene-polyethylene oxide, polyvinylamine-polyethylene oxide, polyglutamic acid-polyethylene oxide, polyacrylic acid and combinations thereof. Further, monomers of the first polymer and/or second polymer, each of which is not bonded to a nucleobase (nucleobase-free monomers), may form a heteropolymer, preferably a heteropolymer selected from the group consisting of polypeptide, polysaccharide, and combinations thereof.

In a preferred embodiment, the mixture does not comprise an organic solvent. Consequently, the mixture may be provided with a lower safety risk, at a lower cost and in a more environmentally friendly way than known prior art mixtures using an organic solvent. It also enables the encapsulation of active substances that are damaged or even destroyed by organic solvents (e.g., certain macromolecular biotherapeutics such as certain proteins).

The mixture according to the invention is also provided for use in medicine, preferably for the thermal release of an active substance, particularly preferably for the thermal release of an active substance for the treatment of cancer.

An aqueous solution comprising a mixture according to the invention is additionally provided in accordance with the invention, wherein the first polymer and the second polymer are at least partially (preferably fully) assembled to micelles or vesicles. In accordance with the invention, the term “solution” is also understood to mean an emulsion and suspension, since the micelles and/or vesicles may also be present emulsified and/or suspended in the water of the solution.

The aqueous solution may be provided, for example, by adding water at room temperature (25° C.) to a mixture according to the invention, incubating the resulting mixture at a temperature above 37° C. (e.g., 40 to 100° C.) for a certain period of time (e.g., 10 to 100 minutes) and then cooling it to a temperature of 37° C. Alternatively, water at a temperature above 37° C. (e.g., 40 to 100° C.) may be added to the mixture according to the invention, incubated for a certain period of time (e.g., 10 to 100 minutes) and then cooled to a temperature of 37° C.

In a preferred embodiment, the aqueous solution does not comprise any organic solvent. The advantage is that the aqueous solution is physiologically more compatible and no damage is caused by organic solvents when the aqueous solution is administered to a living being.

The micelles and/or vesicles of the aqueous solution may have an average hydrodynamic radius, determined by dynamic light scattering, in the range from 30 nm to 300 nm, preferably in the range from 50 nm to 200 nm, optionally in the range from 100 nm to 150 nm. A hydrodynamic radius in this range has proven to be advantageous for the encapsulation and transportation of active substances.

The micelles and/or vesicles of the aqueous solution may comprise an active substance in an interior space (of the micelles and/or vesicles), wherein the active substance is preferably selected from the group consisting of medicinally active substances, cosmetic substances, sensor substances, lubricants, coolants, adhesives and combinations thereof, wherein the active substance is particularly preferably selected from the group consisting of medicinally active substances, fragrances, antiperspirants, UV-protective substances, coolants, lubricants, adhesives and combinations thereof.

Use of the aqueous solution according to the invention for the thermal release of an active substance is also proposed in accordance with the invention, preferably for the release of an active substance selected from the group consisting of medicinally active substances, cosmetic substances, sensor substances, lubricants, coolants, adhesives and combinations thereof, wherein the active substance is particularly preferably selected from the group consisting of medicinally active substances, fragrances, antiperspirants, UV-protective substances, coolants, lubricants, adhesives and combinations thereof.

The subject matter according to the invention will be explained in greater detail on the basis of the following figures and examples, without wishing to limit these to the specific embodiments presented here.

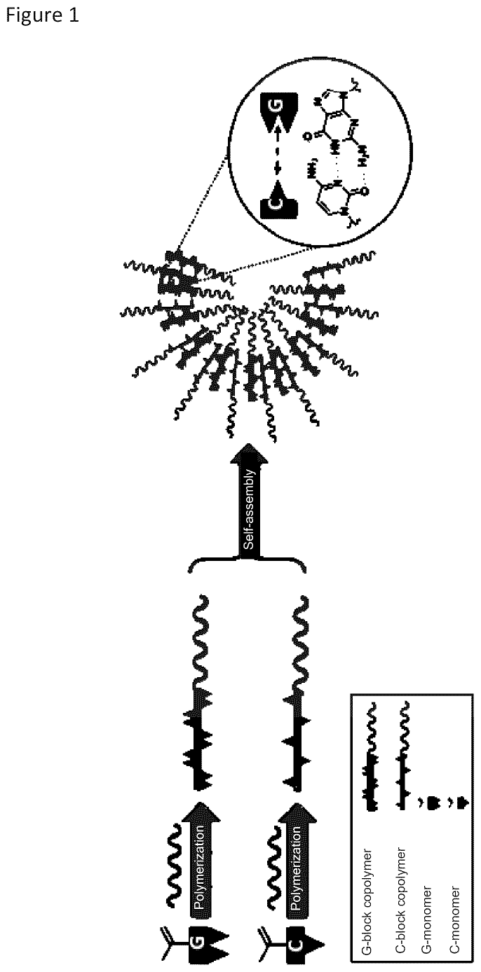

FIG. 1 schematically shows a polymerization and self-assembly (self-organization) of linear first polymers and second polymers formed by complementary nucleoside block copolymers (C and G), which may comprise the mixture according to the invention.

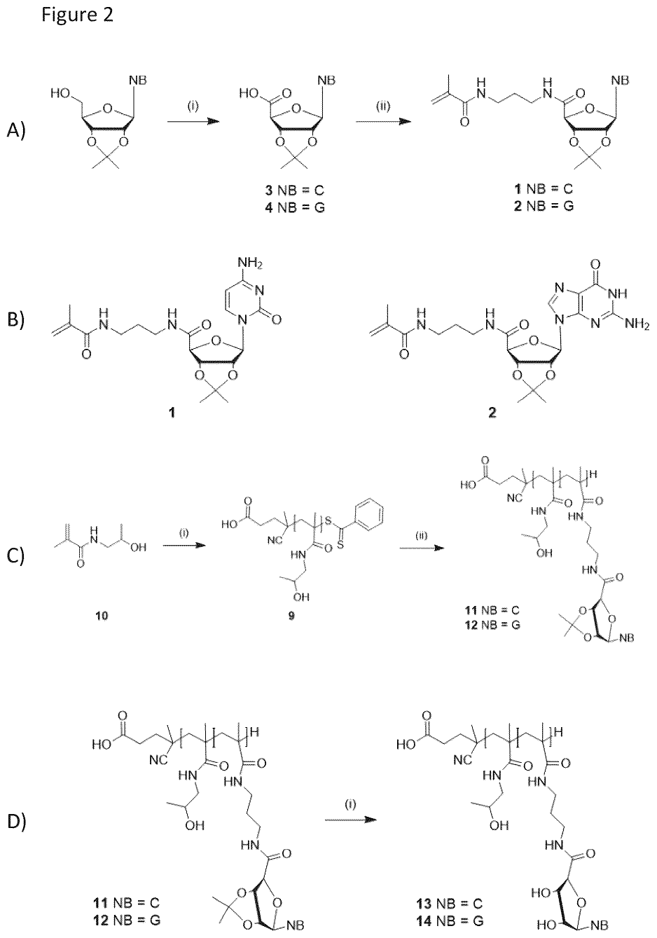

FIG. 2A shows the synthesis of nucleobase (NB) monomer derivatives in a reaction scheme: (i) TEMPO, BAIB, CH₃CN/H₂O, rt, overnight (3: 44%, 4: 98%); (ii) APMA*HCl, CDMT, NMM, MeOH, rt, overnight (1: 44%, 2: 52%).

FIG. 2B shows the chemical structures of ribonucleoside methacrylamides, specifically a cytidine methacrylamide derivative (1) and a guanosine methacrylamine derivative (2), which may be polymerized to produce one end of the first polymer (e.g., polymerization of the cytidine methacrylamide derivative) and one end of the second polymer (e.g., polymerization of the guanosine methacrylamine derivative), wherein the first end comprises monomers each covalently bonded to a nucleobase (=nucleobase-comprising region of the first and/or second polymer).

FIG. 2C shows, in a reaction scheme, the synthesis of pHPMA 9 and nucleoside-based block copolymers pHPMA-b-piCPMA 11 and pHPMA-b-piGPMA 12: (i) ACVA, acetate buffer (pH 5)/EtOH, 70° C., 24 h; (ii) ACVA, DMF/H₂O or 1,4-dioxane/H₂O, 75° C., 24 h. pHPMA may represent a second end of the first polymer and a second end of the second polymer, wherein the second end in each case comprises monomers which are not covalently bonded to a nucleobase (=nucleobase-free region of the first and second polymers). The nucleoside-based block copolymer pHPMA-b-piCPMA 11 may represent a first polymer and the nucleoside-based block copolymer pHPMA-b-piGPMA 12 may represent a second polymer of the mixture according to the invention.

FIG. 2D shows, in a reaction scheme, an acidic deprotection of the acetonide function of pHPMA-b-piCPMA 11 and pHPMA-b-piGPMA 12: (i) TFA, H₂O, rt, 2 h (11: 53%, 12: 81%) to establish OH groups on the C2 atom and the C3 atom of the ribose residue. This measure may increase the solubility of pHPMA-b-piCPMA 11 and pHPMA-b-piGPMA 12 in water.

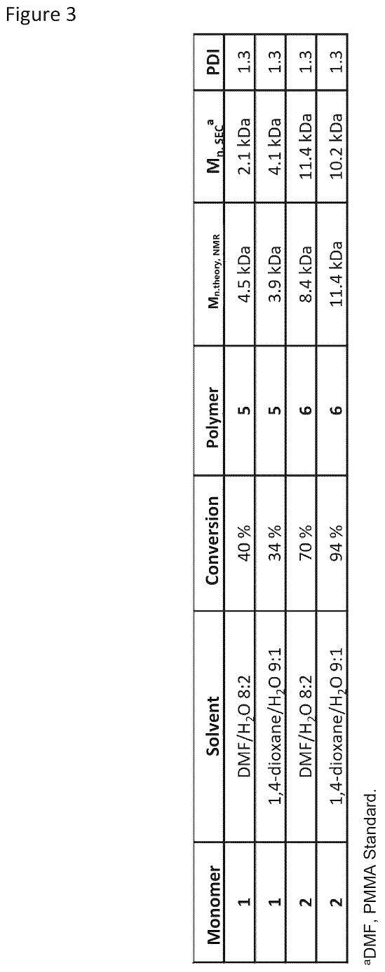

FIG. 3 shows analytical data of piCPMA 5 and piGPMA 6, i.e., of a portion (e.g., piCPMA 5) of a first polymer (e.g., pHPMA-b-piCPMA) and of a portion (e.g., piGPMA 6) of a second polymer (e.g., pHPMA-b-piGPMA) of a mixture according to the invention.

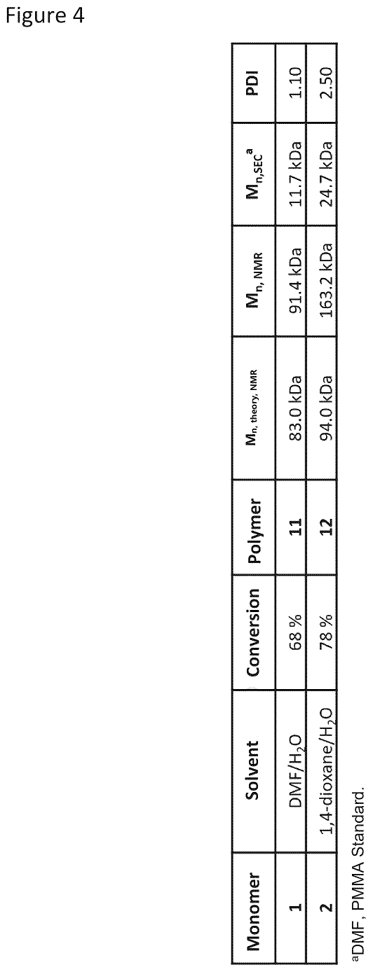

FIG. 4 shows analytical data of pHPMA-b-piCPMA 11 and pHPMA-b-piGPMA 12, i.e., of a first polymer (e.g., pHPMA-b-piCPMA) and of a second polymer (e.g., pHPMA-b-piGPMA) of a mixture according to the invention.

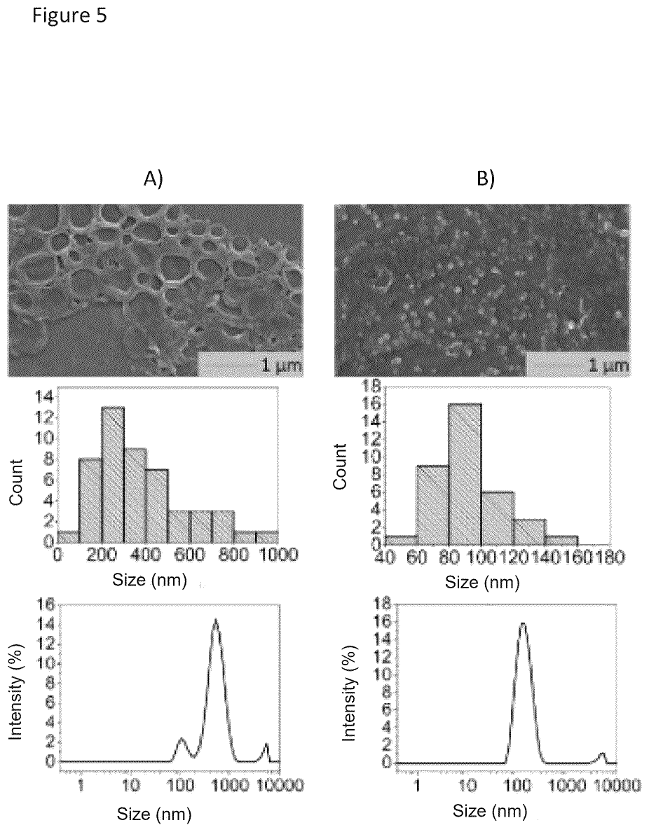

FIG. 5A shows SEM image, size distribution and hydrodynamic size distribution by DLS of pHPMA-b-pCPMA 13.

FIG. 5B shows SEM image, size distribution and hydrodynamic size distribution by DLS of pHPMA-b-pGPMA 14.

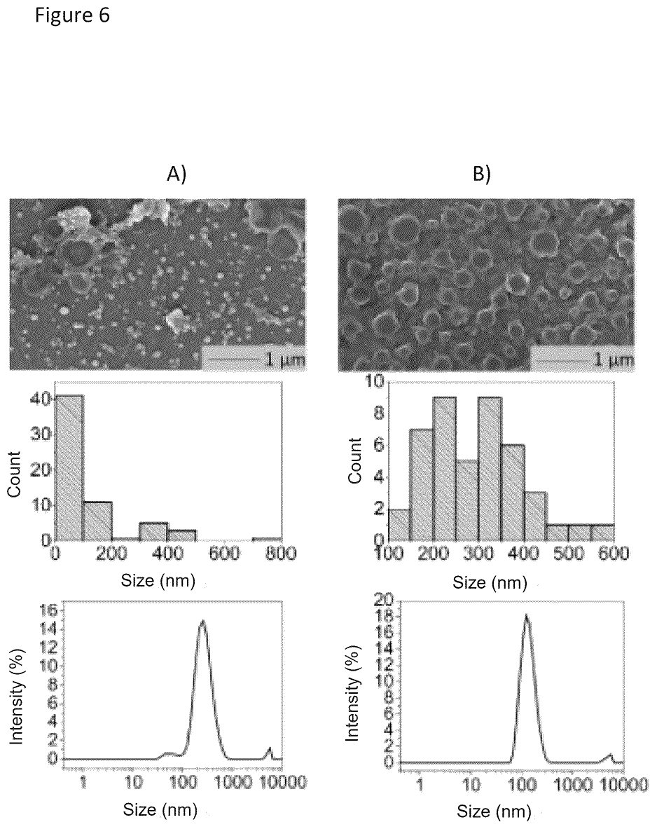

FIG. 6A shows SEM image, size distribution and hydrodynamic size distribution by DLS of a mixture of pHPMA-b-pCPMA 13 and pHPMA-b-pGPMA 14 at room temperature (25° C.) before heating to >37° C. (100° C.).

FIG. 6B shows SEM image, size distribution and hydrodynamic size distribution by DLS of the same mixture from FIG. 5A, only after heating to >37° C. (100° C.) for 30 minutes and subsequent cooling to room temperature (25° C.).

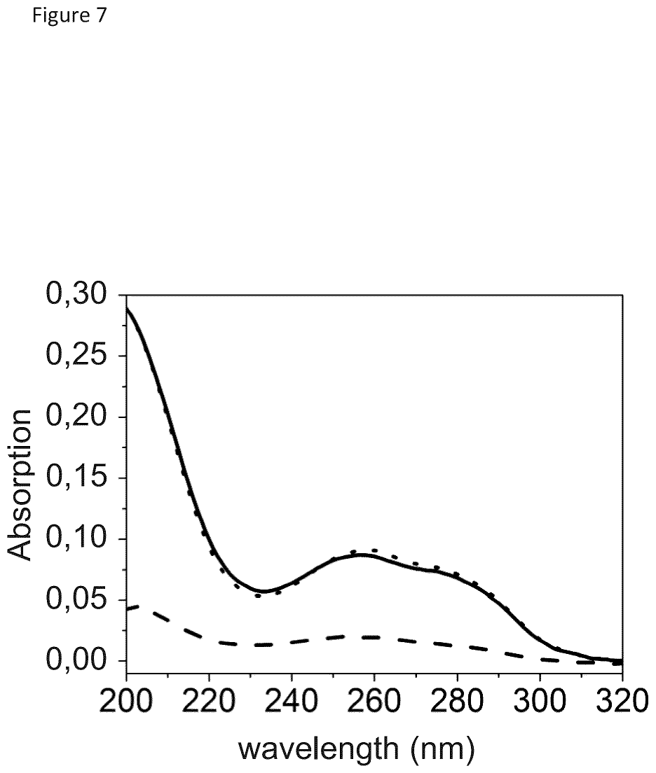

FIG. 7 shows a UV-Vis spectrum of the average of the individual polymer 13 and polymer 14 (line form: - - - -), a mixture of polymer 13 and polymer 14 at room temperature (25° C.) before heating to >37° C. (100° C.) (line form: -) and the mixture of polymer 13 and polymer 14 after heating to >37° C. (100° C.) for 30 minutes and subsequent cooling to room temperature (25° C.) (line form: - - -).

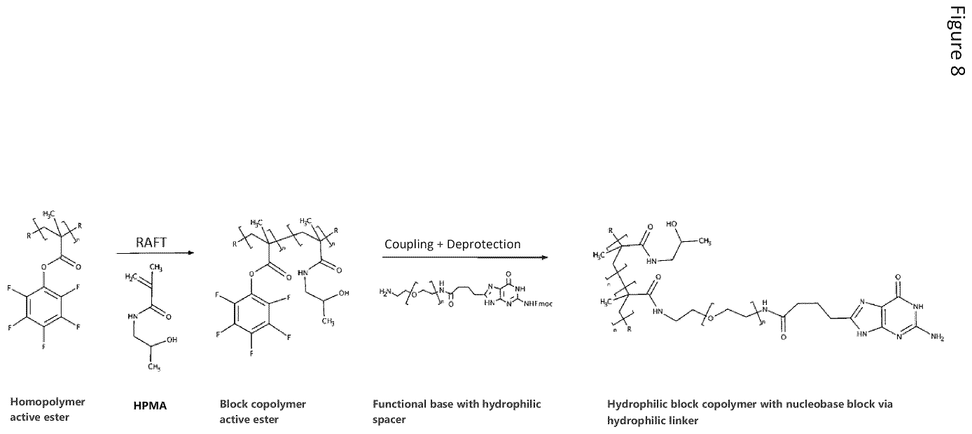

FIG. 8 shows a reaction scheme of another possible RAFT polymerization for producing a first polymer or a second polymer of a mixture according to the invention.

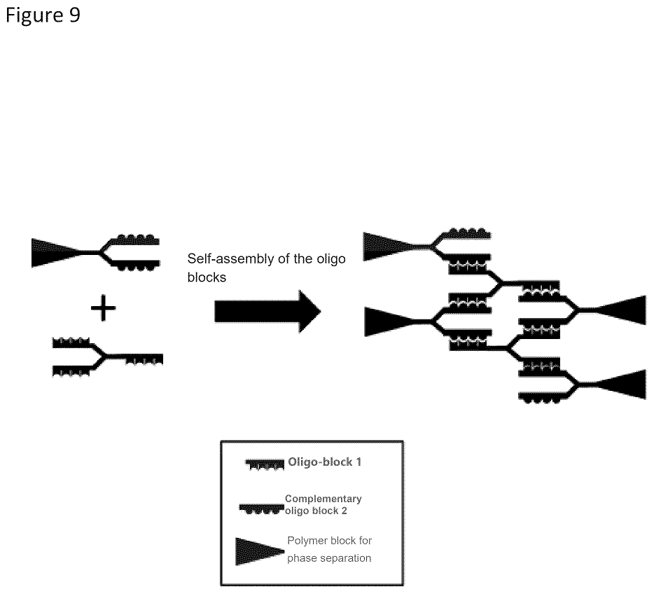

FIG. 9 schematically shows a self-assembly of branched first polymers and second polymers, which may comprise the mixture according to the invention.

### EXAMPLE 1—BASIC PRINCIPLES FOR ASSEMBLING THE POLYMERS OF THE MIXTURE

Israelachvili et al. presented their theory of self-organization of amphiphilic molecules in 1976 and introduced the concept of the critical packing parameter (P_{c}) to predict the formation of supramolecular structures from amphiphilic molecules (Israelachvili, J. N. et al., Theory of self-assembly of hydrocarbon amphiphiles into micelles and bilayers, Journal of the Chemical Society-Faraday Transactions, 72:1525-1568 (1976).

The critical packing parameter is defined as

$${P}_{c} = v / {a}_{0} {\vert}_{c}$$

wherein
v: volume of the hydrophobic portion,
a₀: area occupied by the hydrophilic head group,
l_{c}: contour length of the molecule.

The critical packing parameter thus describes the form of the molecule, which is related to the curvature at the hydrophobic-hydrophilic interface and thus enables a prediction of the self-organized structure

P_{c} values below 0.5 (in particular 0.33) result in strongly curved aggregates such as spherical and cylindrical micelles (single-layer arrangement). P_{c} values of 0.5 to 1 result in the formation of vesicles (two-layer arrangement). P_{c} values of >1 result in the formation of inverse micelles.

In the present case, the first polymer and the second polymer of the mixture according to the invention do not have a hydrophilic region (portion) and a hydrophobic region (portion), but two hydrophilic regions (portions). Strictly speaking, the first polymer and the second polymer of the mixture according to the invention are therefore not amphiphilic molecules.

Nevertheless, the self-assembly theory developed by Israelachvili et al. may also be applied to the first polymer and the second polymer of the mixture according to the invention (see e.g., Polymer Vesicles, D. E. Discher & A. Eisenberg; Science, 297:967-973). In the polymers of the mixture according to the invention, the “hydrophobic” region in the amphiphilic molecules of the theory then corresponds to a region of the first polymer and of the second polymer which comprises the monomers each covalently bonded to a nucleobase. This is the region of the first polymer and the second polymer that leads to an assembly of the first and second polymers so as to form micelles or vesicles via hybridization of complementary nucleobases between the first and second polymers in an aqueous medium at a temperature of 37° C. This favored interaction leads to a microphase separation of the non-functionalized hydrophilic portion and the other hydrophilic portion assembling with its complementary counterpart through the formation of hydrogen bonds and pi-stacking, whereby the two polymer portions are not evenly miscible, but cause a phase separation by segregation, which is responsible for the supramolecular structure formation.

This type of assembly is comparable to the assembly of the hydrophobic region of lipids, which is driven by the hydrophobic effect. In contrast to the assembly of lipids via the hydrophobic effect, however, the assembly of the polymers in the mixture according to the invention can be canceled by raising the temperature to a temperature in the range of >37° C., as a result of which assembled micelles or vesicles disassemble, i.e., lose their micellar or vesicular structure.

The region (portion) of the first polymer and the second polymer that does not have monomers bonded to nucleobases (e.g., a region comprising or consisting of PEG) may be suitable for favoring phase separation. Advantageously, this region (portion) is more hydrophilic than the region (portion) of the first and second polymers comprising the nucleobase-linked monomers.

Since the self-assembly theory developed by Israelachvili et al. may also be applied to the first polymer and the second polymer of the mixture according to the invention, the critical packing parameter for the first polymer and the second polymer of the mixture according to the invention may be defined as:

$${P}_{c} = v / {a}_{0} {\vert}_{c}$$

wherein
v: volume of the region of the polymer that comprises monomers, each of which is bound to a nucleobase (nucleobase-comprising region),
a₀: area formed by the region of the polymer that has no monomers bound to nucleobases (i.e., nucleobase-free region)
l_{c}: contour length of the polymer molecule.

The P_{c} should be substantially the same for the first polymer and the second polymer (deviation preferably at most 10%). Common P_{c} values below 0.5 (in particular 0.33) cause the first and second polymers to assemble so as to form spherical and cylindrical micelles, whereas common P_{c} values in the range from 0.5 to 1 cause the first and second polymers to assemble so as to form vesicles. Common P_{c} values of >1 may cause assembly so as to form inverse micelles. According to the invention, P_{c} values in the range 0.5 to 1 are preferred, since vesicles are formed in this range and the formation of vesicles is a preferred embodiment of the invention.

### EXAMPLE 2—LINKING OF NUCLEOBASES WITH MONOMERS

As nucleobases, nucleosides were used as protected 2′,3′-acetonide forms to address the 5′-position. For stability reasons, methacrylamide-functionalized ribonucleosides were preferred to methacrylate derivatives, which may be synthesized enzymatically. The cytidine (1) and guanosine-based monomers (2) (see FIG. 2B) were synthesized in a two-step process involving oxidation of the primary hydroxyl group and subsequent amide coupling with N-(3-aminopropyl)-methacrylamide (see FIG. 2A). Despite the higher nucleophilicity of the exocyclic —NH₂ group, in contrast to enzymatic esterification, no protective step of this functionality was required.

For the oxidation of commercially available 2′,3′-isopropylidene-cytidine and 2′,3′-isopropylidene-guanosine to their carboxylic acid derivatives, the acetal-protected ribonucleosides were briefly oxidized with TEMPO and BAIB in the presence of NaHCO₃. After filtration of the precipitate, oxidized cytidine (3) was obtained in a yield of 44%, while the yield of oxidized guanosine (4) was quantitative as a white powder (see FIG. 1A). Compounds 1 and 2 (see FIG. 2B) were obtained after amide coupling of compounds 3 and 4 (see FIG. 2A) with N-(3-aminopropyl)-methacrylamide hydrochloride using 2-chloro-4,6-dimethoxy-1,3,5-triazine (CDMT) and N-methylmorpholine (NMM) with a yield of 44% and 52%, respectively (see FIG. 2A). The chemical structure was confirmed by NMR spectroscopy and ESI-MS analyses.

Compound 2 showed lower solubility than compound 1, but both ribonucleoside methacrylamide-based monomers showed adequate solubility in non-polar solvents, such as chloroform and diethyl ether, and in polar solvents such as dichloromethane, acetone and dimethylformamide as aprotic solvents, and water, methanol and ethanol as protic solvents. This solubility property may be explained by the simultaneous formation of hydrogen bonds and hydrophobic parts in a molecule. Due to the high solubility of the two monomer molecules, no further deprotection of the nucleoside monomers was carried out for the polymerizations.

### EXAMPLE 3—PREPARATION OF A NUCLEOBASE-COMPRISING PORTION OF THE POLYMERS

RAFT-mediated polymerization is one of the most important and well-known polymerization techniques involving a radical initiator and a chain transfer agent (CTA). Here, the dithioester-based CTA 4-cyano-4-(phenylcarbonothioylthio)pentanoic acid (CPADB) was selected because it has been described for the polymerization of methacrylamide-based monomers.

Compounds 1 and 2 (see FIG. 2B) were homopolymerized at 75° C. using RAFT polymerization with ACVA as a thermal initiator. Polymerizations of nucleoside-based monomers were carried out with a ratio of [M0]:[CTA0]:[I0] of 75:3:1. The observation of solvent mixtures of 8:2 DMF/H₂O and 9:1 1,4-dioxane/H₂O showed different effects depending on the nucleoside type. The use of a 9:1 1,4-dioxane/H₂O mixture resulted in a high conversion (94%) of the G-based polymer (piGPMA), while the conversion of the cytidine-based polymer (piCPMA) was reduced to 34% with the same solvent mixture. On the other hand, in 8:2 DMF/H₂O the conversion of piCPMA was increased to 40% and that of piGPMA was reduced to only 70%.

The polymerizations of the nucleoside homopolymers and their monomer transformations were determined by comparing the integrals of the typical C-4 protons of piCPMA (δ 4.43 ppm) and piGPMA (δ 4.50 ppm) with the integrals of the monomeric vinyl peaks of iCPMA (δ 5.64 ppm and 5.30 ppm) and iGPMA (δ 6.39 ppm and 5.61 ppm). The theoretical molecular weights (Mn, theory, NMR) are summarized in FIG. 3.

The acetonide protective groups of the homopolymers of both ribonucleosides were removed under acidic conditions with trifluoroacetic acid (see FIG. 2D) to improve the hydrophilicity due to the challenging solubility properties.

### EXAMPLE 4—PREPARATION OF POLYMERS OF MIXTURES ACCORDING TO THE INVENTION

Polymers according to the invention, here specifically block copolymers with nucleosides (pHPMA-b-piCPMA 11 and pHPMA-b-piGPMA 12), were prepared using the RAFT-mediated polymerization technique.

Specifically, a nucleobase-free polymer was first produced. Poly(N-(2-hydroxypropyl)methacrylamide) (pHPMA) was selected due to its biocompatibility. For this purpose, an HPMA macroinitiator was first prepared via RAFT-mediated polymerization according to S. Perrier (50th Anniversary Perspective, RAFT Polymerization—A User Guide, Macromolecules, 2017, 50, 7433-7447). The monomer conversion to pHPMA was 75%, resulting in a theoretical Mn of 7.8 kDa.

Subsequently, compound 1 or compound 2 (see FIG. 2B) was polymerized to the pHPMA via RAFT-mediated polymerization to produce polymers (block copolymers) according to the invention. These were synthesized with a low “liveliness” rate. “Liveliness” is a property that enables the chain to be extended. It indicates how many “living” chains remain intact for further block copolymerization. Low “liveliness” leads to high amounts of dead ends, resulting in nucleobase-based polymers without the phenylic Z group, which may interfere with UV-Vis spectroscopy analysis. The calculated “liveliness” rates were kept low and were 36.0% for polymer 11 and 15.6% for polymer 12.

The polymerization of compound 1 or compound 2 was carried out in the solvent system that was most suitable for the homopolymers: compound 1 in 8:2 DMF/H₂O, compound 2 in 9:1 1,4-dioxane/H₂O. The pyrimidine-based compound 2 led to a higher conversion and thus to a higher molecular weight than the purine-based compound 1 (see FIG. 4). The monomer conversion was determined by comparing the integrals of the monomer peak (1: δ 5.30 ppm; 2: δ 5.61 ppm) with the peak of the nucleoside-based polymer (b-piCPMA δ 4.37 ppm or b-piGPMA δ 6.14 ppm). The monomer conversion for polymer 11 was 68%, while for polymer 12 it was 78% (see FIG. 4).

The two nucleoside-based block copolymers 11 and 12 exhibited low solubilities in water due to the integrated nucleobases.

The block copolymers were removed by acidic deprotection of the acetal functionalities with trifluoroacetic acid (FIG. 2D). The successful deprotection was confirmed by the disappearance of the two singletts in the upfield originating from the acetal protective groups of polymer 11 (δ 1.46 ppm and 1.28 ppm) and polymer 12 (δ 1.48 and 1.33 ppm) in the 1H NMR spectroscopy analysis. By shaking for a total of 2 hours, polymer 13 and polymer 14 were obtained (FIG. 4D), which are polymers according to the invention and which exhibit improved solubility in water.

### EXAMPLE 5—ASSEMBLY OF THE POLYMERS SO AS TO FORM VESICLES

SEM and DLS analyses were performed (FIGS. 5 and 6) to investigate vesicle formation (see FIG. 1) due to hydrogen bond interactions between the purine and pyrimidine functionalities.

The SEM images show that pHPMA-b-pCPMA 13 form large network structures in aqueous solution. These structures result from stronger C-C interactions with a broad size distribution and an average size of 280 nm. This broad size distribution was also determined using DLS with a polydispersity index (PDI) of 0.421. The average hydrodynamic diameter of polymer 13 is 507 nm. However, SEM images of pHPMA-b-pGPMA 14 show small particles due to G-G interactions with an average size of 86 nm. According to the DLS, the size distribution was smaller with an average hydrodynamic diameter of around 144 nm (PDI=0.213) (FIG. 5)

Mixing the two complementary block copolymers resulted in particles with an average hydrodynamic diameter of about 165 nm and a PDI of 0.3. Heating this mixture to 100° C. for 30 minutes and subsequent cooling resulted in a narrower size distribution and a smaller average size of 266 nm and a hydrodynamic diameter of 136 nm (FIG. 6).

This observation could be explained by the breaking of the strong C-C and G-G hydrogen bond at higher temperatures and the restoration of the C-G interactions upon cooling to room temperature (rt). The sonication of polymers 13, 14 and the mixture of both polymers 13, 14 did not lead to any change in morphology, which indicates a high stability.

Nucleobases exhibit strong UV absorption due to hydrogen bonds and n-r interactions. Base pairing interactions of nucleobase derivatives lead to changes in UV-Vis spectroscopy. Spectrophotometric measurements were carried out to investigate the hydrogen bond interactions of the complementary nucleoside-comprising polymers 13 and 14 (FIG. 7).

The UV absorption spectra of the individual polymers 13 and 14 were compared with the spectrum of the mixture after heating. The average values of the individual polymers pHPMA-b-pCPMA 13 and pHPMA-b-pGPMA 14 correspond to the absorption values of the mixture of both polymers at the same concentrations, which is due to the hydrogen bonds of the individual polymers.

After heating the polymer mixture to a temperature of >37° C. (100° C.) for 30 minutes, a decrease in absorption was observed (hypochromism), resulting from the reassembly of the complementary C-G interactions after heating and cooling.

The hypochromism at a wavelength of 260 nm is seen as an indication of dsDNA-like structures, which have a lower absorption compared to ssDNA. This shows that the targeted assembly of polymers 13 and 14 into vesicles via hybridization of their nucleobases was successful.

### EXAMPLE 6—ALTERNATIVE PREPARATION OF POLYMERS OF MIXTURES ACCORDING TO THE INVENTION

In Example 4, a first example was given of how polymers according to the invention, i.e., polymers of a mixture according to the invention, may be prepared. An alternative manufacturing process is described below, which is also based on RAFT-mediated polymerization (see also FIG. 8).

Monomers of (N-(2-hydroxypropyl)methacrylamide (HPMA) are attached to a homopolymer of which the monomers each comprise an active ester, so that a block copolymer is formed, the first block of which is formed by the homopolymer, the monomers of which each comprise an active ester, and the second block of which is formed by poly-(N-(2-hydroxypropyl)methacrylamide (pHPMA). If this block copolymer is now coupled and deprotected with a nucleobase which is bound to a hydrophilic spacer with an amino group, the active ester of the monomers of the first block copolymer reacts with the amino group of the spacer bound to the nucleobase, whereby each nucleobase is bound to a monomer of the first block of the block copolymer via its hydrophilic spacer and via an amide bond. The result is a block copolymer of which the first block consists of monomers, each of which has bound a nucleobase (via a hydrophilic linker) and the second block of which consists of pHPMA.

### EXAMPLE 7—ASSEMBLY OF BRANCHED POLYMERS

The first polymer and the second polymer may each be a branched polymer, i.e., a polymer with at least three arms (e.g., 3-arm PEG or 4-arm PEG).

In this case, it is advantageous if the first polymer and the second polymer comprise the at least six nucleobases, each of which is covalently bonded to a monomer of the polymer, distributed at a first end and at a second end of the first polymer and of the second polymer, respectively. For example, the first polymer comprises six monomers each covalently bonded to a nucleobase at a first end and six monomers each covalently bonded to a nucleobase at a second end (see FIG. 9: oligo block 1) and the second polymer comprises six monomers each covalently bonded to a nucleobase at a first end and six monomers each covalently bonded to a nucleobase at a second end (see FIG. 9: complementary oligo block 2).

The branching in the first polymer and in the second polymer may be realized in such a way that a monomer of the first polymer and of the second polymer, which is not (directly) connected to a nucleobase and is located between the first and second ends of the first and second polymers, respectively (in particular in the middle thereof), is bonded to a further polymer (e.g., PEG), optionally via a spacer (or linker, e.g., a polymer linker). In this case, the other polymer branches off from the first and second polymers, so that the first and second polymers form a kind of “forked structure” (see FIG. 9). The further polymer is advantageously suitable for causing phase separation after bonding of the first polymers to the second polymers in an aqueous phase at a temperature in the range of 37° C. (see FIG. 9: polymer block for phase separation).

If the mixture according to the invention comprises two branched polymers, it is advantageous for assembly so as to form micelles or vesicles if the volume fraction of the region of the first polymer and of the second polymer which assembles in aqueous solution (i.e., of the region comprising the monomers which are each bonded to a nucleobase) is as similar as possible to the volume fraction of the region of the first polymer and of the second polymer which does not assemble in aqueous solution (i.e., of the region comprising the monomers which are in each case not bonded to a nucleobase).

The first polymer and the second polymer may each comprise a 4-arm PEG (e.g., having a molar mass of 5000 g/mol), wherein at least two arms of the 4-arm PEG comprise the monomers (preferably at their ends) which are in each case covalently bonded to a nucleobase. These monomers may represent an oligomer consisting of monomers sequentially bound to nucleobases (e.g., each arm has four or six such monomers). The oligomer may be formed as an oligonucleotide.

1-15. (canceled)

16. A mixture, comprising
a) a first polymer comprising at least six nucleobases, wherein one nucleobase is covalently bonded to one monomer of the first polymer, respectively; and
b) a second polymer comprising at least six nucleobases, wherein one nucleobase is covalently bonded to one monomer of the second polymer, respectively, wherein the at least six nucleobases of the second polymer are complementary to the at least six nucleobases of the first polymer;
wherein the first polymer and the second polymer are capable of not assembling to micelles or vesicles via their mutually complementary nucleobases, in an aqueous solution
at a temperature of <37° C., by forming hydrogen bonds between their complementary nucleobases, and
at a temperature of >37° C., due to the absence of hydrogen bonds between their complementary nucleobases.

17. The mixture according to claim 16, wherein the mixture further comprises an active substance, wherein the active substance is selected from the group consisting of medicinally active substances, cosmetic substances, sensor substances, lubricants, coolants, adhesives, and combinations thereof.

18. The mixture according to claim 16, wherein the
i) first polymer comprises at least 20 monomers; and/or
ii) second polymer comprises at least 20 monomers.

19. The mixture according to claim 16, wherein the
i) first polymer comprises at least ten nucleobases, wherein one nucleobase is covalently bonded to one monomer of the first polymer, respectively; and
ii) second polymer comprises at least ten nucleobases, wherein one nucleobase is covalently bonded to one monomer of the second polymer, respectively, wherein the nucleobases of the second polymer are complementary to the nucleobases of the first polymer.

20. The mixture according to claim 16, wherein, in the
i) first polymer, a maximum of three monomers, which is not covalently bonded to a nucleobase, is arranged between two successive monomers each bonded to one nucleobase; and/or in the
ii) second polymer, a maximum of three monomers, which is not covalently bonded to a nucleobase, is arranged between two successive monomers each bonded to one nucleobase.

21. The mixture according to claim 16, wherein the nucleobases in the
i) first polymer are nucleobases comprising cytosine, isocytosine and/or adenine; and/or
ii) second polymer are nucleobases comprising guanine, isoguanine and/or thymine.

22. The mixture according to claim 16, wherein the nucleobases of the
i) first polymer are bonded to the monomer of the first polymer via a ribose residue,
ii) second polymer are bonded to the each one monomer of the first polymer via a ribose residue.

23. The mixture according to claim 16, wherein the monomers of the first polymer and/or of the second polymer, which are each bonded to one nucleobase,
i) each comprises a residue selected from the group consisting of amino acid residue, sugar residue, ribose phosphate residue, methacrylamide residue, and combinations thereof,
and/or
ii) form a homopolymer, wherein the homopolymer is selected from the group consisting of polypeptide, polysaccharide, polyribose phosphate, and polymethacrylamide, and/or
iii) are covalently bonded to the nucleobase via a bond, optionally via a ribose residue, which is selected from the group consisting of ester bond, thioester bond, ether bond and amide bond.

24. The mixture according to claim 16, wherein monomers of the first polymer and/or second polymer, which are each not bonded to a nucleobase,
i) each comprises a residue selected from the group consisting of (N-(2-hydroxypropyl)methacrylamide residue, vinylamine residue, butadiene residue, ethylene oxide residue, acrylic acid residue, amino acid residue, and sugar residue; and/or
ii) form a homopolymer, a block copolymer, or a heteropolymer.

25. The mixture according to claim 16, wherein the mixture does not comprise an organic solvent.

26. A method of treating a patient comprising administering a mixture according to claim 16 to the patient, whereby an active substance is thermally released to the patient.

27. An aqueous solution comprising a mixture according to claim 16,
wherein the first polymer and the second polymer are at least partially assembled to micelles or vesicles.

28. The aqueous solution according to claim 27, wherein the micelles and/or vesicles have an average hydrodynamic radius, determined by dynamic light scattering, in the range from 30 nm to 300 nm.

29. The aqueous solution according to claim 27, wherein the micelles and/or vesicles comprise an active substance or active substances in an interior space, wherein the active substance or active substances is/are selected from the group consisting of medicinally active substances, cosmetic substances, sensor substances, lubricants, coolants, adhesives, and combinations thereof.

30. A method of thermally releasing an active substance or active substances to a substrate comprising applying to the substrate an aqueous solution according to claim 29.

31. The method according to claim 30, wherein the active substance or actives substances is/or selected from the group consisting of medicinally active substances, cosmetic substances, sensor substances, lubricants, coolants, adhesives, and combinations thereof.
